(19) United States
(12) Patent Application Publication
(10) Pub. No.: US 2023/0364374 A1
(43) Pub. Date: Nov. 16, 2023

(54) Oxygen Supply Device With FiO2 Control

(71) Applicant: Telesair, Inc., Irvine, CA (US)

(72) Inventors: Yong Liu, Irvine, CA (US); Qing Wang, Irvine, CA (US)

(21) Appl. No.: 17/744,618

(22) Filed: May 13, 2022

(51) Int. Cl. A61M 16/12; A61M 16/10; A61M 16/00

(52) U.S. Cl. CPC A61M 16/125; A61M 16/101; A61M 16/0066; A61M 2016/1025; A61M 2205/3337; A61M 2205/3386; A61M 2205/3389; A61M 2202/0208

(57) ABSTRACT

Embodiments provide an oxygen supply device comprising an oxygen production unit, a control unit, an air blower and a gas conditioning unit. The oxygen production unit comprises multiple adsorption units including a first adsorption unit and a second adsorption unit; the air blower is configured to receive ambient air and oxygen generated by the oxygen production unit and output blended air at a desired FiO2 ratio. The gas conditioning unit is connected to the air blower and is configured to generate conditioned air using the blended air from the air blower. The control unit is configured to control the first and second adsorption unit to operate independently from each other such that first and second adsorption units operate alternatively; and independently control a flow rate of the oxygen generated by the oxygen generation unit and a flow rate of ambient air into the air blower.

### FIELD OF THE INVENTION

The present disclosure relates to oxygen supply device designed and configured for non-professional use environments such as at a patient's home.

### BACKGROUND OF THE INVENTION

A medical breathing machine is used to help lungs work. Fundamentally, a medical breathing machine helps a patient to get oxygen into the lung, and to remove carbon dioxide from the body. One of the most widely used breathing machine is high flow nasal canula (HFNC). High Flow Nasal Cannula (HFNC) is one kind of oxygen supply device providing oxygen therapy to respiratory function comprised patients and is recommended by the World Health Organization (WHO) and US National Institutes of Health (NIH) as the first device to treat COVID-19 patients. SARS-CoV-2 virus which causes highly contagious COVID-19. Operating HFNC for COVID-19 patients creates enormous risk to health care workers. Over 38,000 healthcare workers were infected by SARA-VoV-2 in Los Angeles hospitals alone.

HFNC machines help patients suffering from limited and impaired breathing capability. Typically, HFNC machines deliver heated and humidified oxygen enriched air flow to patients through a nasal canula. The flow rate of the oxygen air enriched by the HFNC machines is normally higher than the flow rate of normal inspiration air flow rate of a healthy person of the same age group. Clinical results indicate that HFNC is helpful to those patients who suffer from a variety of indications, including hypoxemic respiratory failure due to pneumonia, post-extubation, pre-oxygenation prior to intubation, acute pulmonary edema, etc.

An oxygen supply device is a device that supplies an oxygen-enriched product gas stream. An oxygen supply device can be used in health care institutions to supply oxygen to patients for treatment of breathing-related disorders such as asthma, pneumonia, respiratory distress syndrome, bronchopulmonary dysplasia, chronic obstructive pulmonary disease.

### SUMMARY OF THE INVENTION

In accordance with the present disclosure, an integrated oxygen supply device is provided. In some embodiments, the oxygen supply device in accordance with the present disclosure comprises an oxygen generation unit, a gas conditioning unit, a gas delivery unit, a control unit and/or any other components. In those embodiments, oxygen generation unit is configured to receive ambient air and produce concentrated oxygen. In one embodiment, the oxygen generation unit comprises an oxygen concentration module configured to receive pressure regulated air and generate concentrated oxygen. In that embodiment, the oxygen concentration module comprises one or more of an adsorption unit configured to produce concentrated oxygen by absorbing oxygen from the pressure regulated air and releasing nitrogen in the pressure regulated air. In one example, the oxygen concentration module comprises multiple adsorption units such that they can be configured to operate in parallel or alternatively.

In some embodiments, the control unit is configured to control the oxygen concentration module to set one or more of the adsorption units into a working state or an off state. In the working state, the adsorption unit is configured to separate the oxygen and the nitrogen in the pressure regulated air; and in the off state, the adsorption unit is configured to release concentrated oxygen to an oxygen storage module of the oxygen concentration module and the nitrogen to an environment surrounding the oxygen supply device in accordance with the present disclosure.

Other objects and advantages of the invention will be apparent to those skilled in the art based on the following drawings and detailed description.

### BRIEF DESCRIPTION OF THE DRAWINGS

FIG. 1 illustrates generally an integrated oxygen supply device in accordance with the present disclosure.

FIG. 2 illustrates an example an oxygen supply device shown in FIG. 1.

FIG. 3 illustrates an example of an oxygen generation unit shown in FIG. 2.

FIG. 4 illustrates an example of an oxygen concentration module shown in FIG. 3.

FIG. 5 illustrates another example of the oxygen generation unit shown in FIG. 2.

FIG. 6 illustrates one example of a method for controlling the oxygen concentration module shown in FIG. 4 in accordance with the present disclosure.

FIG. 7 illustrates one example of a gas conditioning unit shown in FIG. 2.

FIG. 8 illustrates another example implementation of an integrated oxygen supply device in accordance with the present disclosure.

FIG. 9 illustrates yet another example implementation of an integrated oxygen supply device in accordance with the present disclosure.

FIG. 10 illustrates an example computer system that can used to implement various embodiments described and illustrated herein.

### DETAILED DESCRIPTION

The following disclosure provides many different embodiments, or examples, for implementing different features of the provided subject matter. Specific examples of components and arrangements are described below to simplify the present disclosure. These are, of course, merely examples and are not intended to be limiting. For example, the formation of a first feature over or on a second feature in the description that follows may include embodiments in which the first and second features are formed in direct contact, and may also include embodiments in which additional features may be formed between the first and second features, such that the first and second features may not be in direct contact. In addition, the present disclosure may repeat reference numerals and/or letters in the various examples. This repetition is for the purpose of simplicity and clarity and does not in itself dictate a relationship between the various embodiments and/or configurations discussed. For a particular repeated reference numeral, cross-reference may be made for its structure and/or function described and illustrated herein.

Patients with moderate to severe COVID-19 are often present with pneumonia that could lead to hypoxemia. High flow nasal cannula (HFNC) is recommended by the World Health Organization (WHO) and National Institutes of Health (NIH) as a way to provide supplemental oxygen for COVID-19 patients who are hypoxemic. HFNC can provide humidified and body-temperature oxygen from 10-80 L/min to meet patients' demand, and decrease patients' discomfort and work of breathing. It also improves ventilation and oxygenation through the washout of nasopharyngeal dead space. When compared to conventional oxygen therapy or Noninvasive Positive Pressure Ventilation (NIPPV), HFNC has been demonstrated to decrease 90-day mortality and intubation rate for patients with acute hypoxemic respiratory failure.

As a patient's condition changes with time; the settings of HFNC also need to be adjusted accordingly. Due to the nature of high gas flow, both the WHO and NIH categorized HFNC as an aerosol generating procedure that is defined as a high-risk exposure for HCWs when used on a COVID-19 patient; it becomes a critical battlefront to ensure the safety of HCWs. Studies have shown that frontline HCWs are at least 3 times more likely to be infected. The danger to HCWs is worse when we have limited supplies of PPE. Compared with the HCWs who do not have exposure to patients with COVID-19, HCWs who have inadequate or reused PPE with exposure to patients with COVID-19 have 5-6 times higher infection rate In Los Angeles County, the most populous county in the United States, over 38,000 HCWs and first responders have been confirmed with COVID-19 as of March 2021. COVID-19 pandemic has been a tremendous challenge to our societies as a whole, but hospitals and our frontline HCWs have been under unprecedented stress because of the overwhelming number of COVID-patients and highly contagious nature of COVID-19. During COVID-19 pandemic, different waves of infection in different countries have been happening in different time. Thus, if HFNC treatment can be provided in-home, some or all of the above-mentioned risks can be reduced or avoided.

One issue with a current oxygen supply device is that it is not optimal for home use to achieve effective HFNC treatment. A reason that HFNC treatment currently is not assessable to patients at home is a requirement of excessive oxygen supply for effectively treating the patients. In some cases, effective HFNC treatment may need a flow rate at 40 liter per minute (LPM), and fraction of inspired oxygen (FiO2) at 40%. High pressure oxygen sources such as compressed gas cylinders or fixed medical oxygen plumbing systems (e.g., oxygen tanks) are conventional means for supplying such a level of enriched oxygen to a patient. However, oxygen tanks are expensive, difficult to handle, hard to move and thus are not typically suitable for home use.

As mentioned above, for an effective HFNC treatment, a flow rate of 40 LPM or higher oxygen supply at 40 FIO2 (40/40) is desired. This means an oxygen source of capable of supplying 11 LPM at 90% FiO2. As the air separation materials used in the oxygen generation unit decay and lose separation efficiency from 90% to 80%, a 13 LPM oxygen generation unit is desirable to consistently meet the 40/40 requirement. Take into consideration of the material decay, an oxygen generation unit at 13 LPM at 90% FiO2 is desired. (note: 16 lpm 90% oxygen source may be required for 40% FiO2 at 50 lpm flow rate(40/50) based on calculation, taken into consideration of material decay)concentrated oxygen is desired to facilitate such a treatment. However, existing portable oxygen generation devices typically can only generate around 2-5 LPM oxygen from ambient air due to size and/or cost limitations. While some portable oxygen concentrators are commercially available to generate around 10 LPM oxygen, it is still short of the 13 LPM desired for the 40/40 oxygen supply mentioned above. In addition, the existing art is expensive, bulky and electrically power hungry for home applications. Thereby, a challenge for implementing effective HFNC treatment in a non-professional environment such as in a patient's home is how to generate and deliver oxygen, for example, at 40LPM/40FIO2, using an oxygen supply device with a reasonable size and affordable.

One insight provided by the present disclosure is an integrated oxygen supply device comprising an oxygen generation unit, a gas conditioning unit, a gas delivery unit, a control unit, and/or any other components. It should be understood the integrated oxygen supply device in accordance with the present disclosure is a single device having a housing, within which the oxygen generation unit, the gas conditioning unit, the gas delivery unit, and the control unit are located. Some considerations for the integrated oxygen supply device in accordance with the present disclosure include form factors, costs (affordability), power consumption, efficiency, effectiveness, and/or any other considerations. As mentioned above, for achieving a relatively small form factor of the integrated oxygen supply device in accordance with the present disclosure, a small or reasonably sized oxygen source is desired, such as an air separation unit capable of generating 13 LPM oxygen.

With such an oxygen source in the integrated oxygen supply device, measures or design choices are to be adopted to make up for the above-mentioned LPM gap for achieving an effective treatment. One insight provided by the present disclosure is that flow of an oxygen output from the air separation unit in the integrated oxygen supply device can be controlled. Other considerations are also explored by the inventor(s) to reduce or close this LPM gap. Insights into the other considerations are culminated into other disclosures to enable HFNC treatment at a non-professional environment such as a patient's home.

In U.S. patent Ser. No. 11/135,390, insights are provided to control an oxygen provider device based on patient's measurement data. There, an aim of such a control is to accurately determine a volume for high concentration oxygen as needed by a patient and adjust the volume dynamically based on the determination so to achieve a therapeutic effect. U.S. patent Ser. No. 11/135,390 is hereby incorporated by reference in its entirety.

In accordance with the present disclosure, in some embodiments, the oxygen generation unit comprises a compressor, a cooling module, a pressure regulator, an oxygen concentration module, an oxygen storage module, and/or any other components. One insight provided by the present disclosure is that the oxygen concentration module can comprise multiple adsorption units that can be controlled to work in parallel or alternatively. In such a design, an oxygen production efficiency is improved. For example, in one embodiment, the oxygen concentration module comprises two adsorption units controllable to work alternatively such that when one of the adsorption units is working to separate oxygen and nitrogen in the received air, the other adsorption unit is set to an off state to release absorbed nitrogen into an environment. In that example, when the current working adsorption unit is saturated with nitrogen, it is controlled to be switched to the off state to release the nitrogen to the environment, and the other adsorption unit is set to the working state to separate oxygen and nitrogen in the received air. In that example, the timing of such a control of the adsorption units is controlled by the control unit. In that example, one or more sensors are arranged at and/or around the oxygen concentration module to monitor a concentration level of the oxygen produced by the working adsorption unit. Based on the monitored concentration level, the control unit can determine whether to switch the working adsorption unit to the off state, and switch the off adsorption unit to the working state.

For achieving one or more of advantages mentioned above, in accordance with the present disclosure, an integrated oxygen supply device that is configured to generate oxygen continuously and release the oxygen non-continuously is provided. The delivery of the oxygen produced by integrated oxygen supply device to a patient, in those embodiments, is controlled. In some embodiments, the control of the delivering oxygen is according one or more breathing patterns. The breathing pattern(s) may or may not be a current breathing pattern of the patient. For example, in one embodiment, the breathing pattern is a predetermined breathing pattern with a specified inspiration period followed by a specified expiration period. The specifications of the inspiration and expiration periods in the predetermined breathing pattern, in that embodiment, are configured to facilitate a 40 LPM oxygen supply to the user for the HFNC treatment.

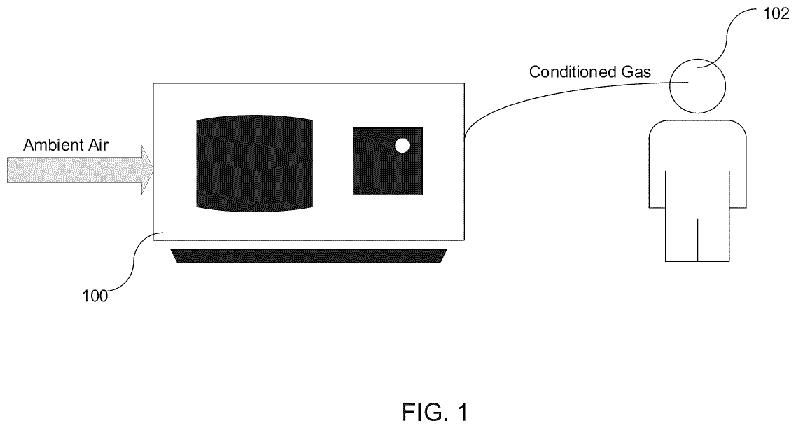

FIG. 1 illustrates generally an integrated oxygen supply device in accordance with the present disclosure. The integrated oxygen supply device 100 in FIG. 1 can use ambient air as an input gas and supply conditioned gas to a patient 102 for a treatment. Examples of the conditioned gas can include pressurized, high flow, heated, and/or humidified gas with a concentration level of oxygen. As mentioned above, an advantage of the integrated oxygen supply device 100 shown in FIG. 1 is that it is a single integrated device that can produce the conditioned gas to the patient 102 and can be used in a non-professional setting such as the patient 102's home due to its form factor and affordability for such a use.

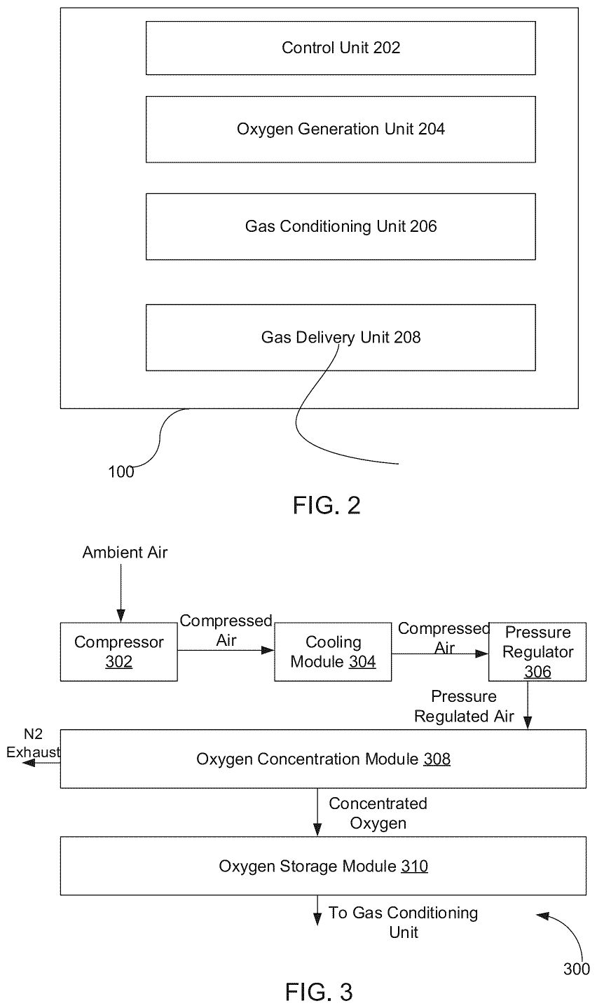

FIG. 2 illustrates an example of the integrated oxygen supply device shown in FIG. 1. As shown, in this example, the integrated oxygen supply device 100 includes a control unit 202, an oxygen generation unit 204, a gas conditioning unit 206, a gas delivery unit 208, and/or any other components. The control unit 202 is configured to control various operations of the integrated oxygen supply device 100. In particular, control unit 202 is configured to control one or more operation aspects of the oxygen generation unit 204, one or more conditions of conditioned gas generated by the gas conditioning unit 206, one or more operations of the gas conditioning unit 206, an oxygen delivery timing to the patient 102 by the gas conditioning unit 206, and/or any other aspects of the integrated oxygen supply device 100.

As will be appreciated by those skilled in the art, control unit 202, may be physically implemented using a software-controlled microprocessor, hard-wired logic circuits, or a combination thereof. For example, the control unit 202 may be implemented as a microprocessor configured to execute one or more software algorithms, including timing control, gas mixing control, gas conditioning processes of various embodiments described herein, in conjunction with a memory (not shown), to provide various functionalities of integrated oxygen supply device 100. That is, the control unit 202 may include a nonvolatile memory for storing executable software code that allows it to perform the various functions of integrated oxygen supply device 100 and various processes, discussed herein.

The oxygen generation unit 204 is configured to receive ambient air, separate oxygen, and nitrogen in the received ambient air to produce concentrate oxygen, and output the concentrated oxygen to the gas conditioning unit 206. In various embodiments, the oxygen generation unit 204 is controllable to store oxygen and output the concentrated oxygen upon a desired timing or frequency. In those embodiments, the oxygen generation unit 204 is capable of outputting concentrated oxygen at a range between 40%-98% oxygen concentration.

The gas conditioning unit 206 is configured to receive the concentrated oxygen output by the oxygen generation unit 204, and ambient air, and output conditioned gas using the received concentrated oxygen and ambient air. In various embodiments, the conditioned gas output by the gas conditioning unit 206 is conditioned by heating, humidifying, pressurizing and/or any other conditioning processes.

The gas delivery unit 208 is configured to receive conditioned gas and facilitate delivery of the received blended gas to the patient 102. In various embodiments, the gas delivery unit 208 is configured to facilitate the delivery of the received conditioned gas according to a pulsing pattern such that the blended gas is delivered to the patient in a controlled timing. It should be understood, not limited to a method where gas is delivered to the patient 102 based on the patient 102's inhale and exhale rhythm, the method employed by the gas delivery unit 208 can include a pulsing pattern predetermined for the patient 102. This can help the patient 102 adjust or adapt to a level desired for the patient 102. In addition, this can help improve oxygen LPM delivered to the patient 102 because the blended gas is not released to the patient 102 continuously.

With the integrated oxygen supply device 100 in accordance with the present disclosure having been generally described, attention is now directed to FIG. 3, where an example 300 of an oxygen generation unit 204 is illustrated. As can be seen, in this example, the example 300 of the oxygen generation unit 204 includes a compressor 302, a cooling module 304, a pressure regulator 306, an oxygen concentration module 308, an oxygen storage module 310, and/or any other components.

In this example, as shown, the compressor is configured to receive ambient air, compress the ambient air to achieve a desired pressure and/or flow rate to obtain compressed air. In implementation, pressure sensors and flow sensors can be applied in the compressor 302 to detect whether pressure and flow rate of the compressed air has reached the desired level. Feedback control mechanisms can be applied in the compressor 302 to ensure the desired pressure and flow rate is achieved. In some embodiments, the control unit 202 is configured to control one or more operation aspects of the compressor 302. For example, the control unit 202 can be configured to control the compressor 302 to compress the ambient air to a preset pressure and/or flow rate. In one instance, the compressor 302 is controlled by the control unit 202 to compress the air to 50 Pounds per Square Inch (PSI).

In this example, the cooling module 304 is used to cool the compressed air output by the compressor 302 to reach a desired temperature level for the air. In various implementation, the cooling module 304 comprises a cooling coil configured to cool the compressed air continuously through heat exchange. In those implementation, the heat exchange process facilitated by the cooling module 304 is controlled by the control unit 202. For instance, the control unit 202 is configured to control the desired temperature level. In that instance, a temperature sensor is employed to provide a feedback mechanism such that temperature readings of the air are s monitored by the control unit 202 for determination whether the desired temperature level has reached. (Did we disclose the heat exchange implementation? For example, the heat released from 304 can be used to raise the temperature of the humidification unit. This is a unique advantage coming from the integration solution of this device. Other sources of heat may be 302, 502, etc). By utilizing the heat from oxygen generation unit, we may also claim to be able to reduce the size of the heater and the power consumption of the humification unit to in the implementation, to reduce the total size and power of the device. It would be nice if we can semi-quantify this.)

The pressure regulator 306 is configured to regulate the pressure and/or the flow rate of the compressed air for input to the oxygen concentration module 308. In some embodiments, the pressure regulator 306 is used to regulate the pressure of the compressed air at a more or less consistent pressure level, such as 50 PSI or 45 PSI, and a consistent flow rate level, such as 90 liter per minute (LPM). For example, the pressure regulator 306 can be configured to ensure the compressed air supplied to the oxygen concentration module 308 is regulated at 50 PSI. This can ensure efficient operation of the oxygen concentration module 308 to produce concentrated oxygen from the compressed air by absorbing nitrogen.

The oxygen concentration module 308 is configured to produce concentrated oxygen from the compressed air and release nitrogen from the compressed air. In implementation, the oxygen concentration module 308 comprises one or more of an adsorption unit configured to separate oxygen from the nitrogen in the compressed air received by the oxygen concentration module 308. In that implementation, the adsorption unit comprises a porous material, such as Zeolite, a MOF structure, a COF structure, and/or any other types of porous material. The porous material in those embodiments is employed to absorb nitrogen from the compressed air so to separate the oxygen from the nitrogen in the compressed air. In one embodiment, the nitrogen content in the compressed air is absorbed when the compressed air flows through the porous material in the oxygen concentration module 308. In that embodiment, because the nitrogen content in the compressed air is absorbed and released through the N2 exhaust shown, enriched oxygen air or concentrated oxygen is produced. In implementation, the release of absorbed nitrogen can be controlled. For example, the absorbed nitrogen is released when the porous material has reached a predetermined saturation level. In implementation, the release of the absorbed nitrogen can be controlled by changing a pressure and/or temperature of the porous material.

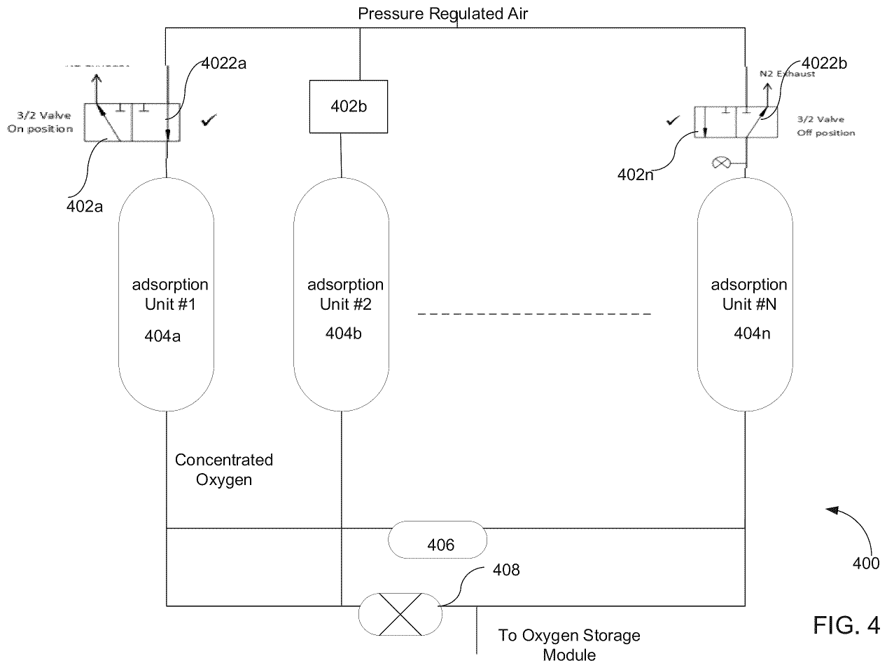

FIG. 4 illustrates one example 400 of the oxygen concentration module 308 in accordance with the present disclosure. As can be seen, the example 400 of the oxygen concentration module 308 comprises multiple adsorption units 404 controllable to operate in parallel and/or alternatively. In this example, a given adsorption unit 404 is connected to a corresponding switch valve 402. For instance, the adsorption unit 404a is connected to the switch valve 402a, the adsorption unit 404b is connected to the switch valve 402b, and the adsorption unit 404n is connected to the switch valve 404n as shown.

Attention is now directed to switch valve 402a for description of a working state of the adsorption unit #1 in this example. As shown, the switch valve 402a is in a first state where a circuit 4022a is closed to a first position to facilitate the pressure regulated air from the pressure regulator 306 to the adsorption unit #1 404a. In the first state, the switch valve 402a also has a position where the nitrogen exhaust is closed. Thus, in the first state, the switch valve 402a allows the pressure regulated air to pass into the adsorption unit #1 404a for concentrated oxygen production.

In some implementation, the switch valve 402a is controlled by the control unit to switch into the first state (e.g., the first circuit 4022a is closed and the N2 exhaust is closed) periodically. In one embodiment, the switch valve 402a is controlled to switched into the first state every 10 seconds to be in the first state for 5 seconds. For instance, at a 0th second the switch valve 402a is controlled to be swept into the first state and remain in the first state for 5 seconds, at the 5th second, the switch valve 402a is controlled to be swept into a second state and remain in the second state for 5 seconds, and at the 10^{th} second, the switch valve 402a is controlled to be swept into the first state; and so on.

Attention is now directed to the switch valve 402n, where the switch valve 402n is set to the second state. In the second state, the first circuit 4022b is in a second position such that the N2 exhaust is open to facilitate release of the nitrogen from the adsorption unit #2 404n, and path for the pressure regulated air to the adsorption unit #n 404n is open such that the pressure regulated air cannot flow into the adsorption unit #n 404n.

In the first state, the adsorption unit #1 404a absorbs the nitrogen from the incoming air at around, for example, 50 PSI. As mentioned, the adsorption unit #1 404a can be set to the first state for a period, such as five seconds. During this period, concentrated oxygen is produced and is released to the oxygen storage module 310 for storage. In the second state, the adsorption unit #n 404n release the absorbed nitrogen through the N2 exhaust to the environment for a period, such as the 5 seconds. Then, at the 5^{th} second, the adsorption unit #1 404a is set to the second state and the adsorption unit #n 404n is set to the first state. In this way, the adsorption unit #1 404a and the adsorption unit #n 404n operate alternatively to improve oxygen production efficiency.

In some embodiments, the individual adsorption units are coordinated by the control unit 202 to work in parallel and/or alternatively to achieve one or more oxygen production goals. In one embodiment, a given adsorption unit, such as the adsorption unit #1 404a comprises a pair of sieve beds. However, this should not be understood limiting. It is contemplated that the adsorption unit in an integrated oxygen supply device in accordance with the present disclosure may comprise any number of sieve beds ranging from 1 to any number. However, it is also understood, a typical adsorption unit in a PSA system comprises a pair of sieve beds for producing oxygen in an efficient manner.

In accordance with the present disclosure, the individual adsorption beds are controllable, for example by the control unit 202, to be switched on/off at any given time independent from each other. As mentioned, for example, turning on adsorption unit #1 404a can turn on the pair of sieve beds therein. Thus, in that example, at a given time, an even number of sieve beds in the adsorption units can be turned on to produce oxygen. (so each unit such as 404a, has 2 subunit or 2 seive bed? if this is the concept, we should clarify in the text.) (why “even” number of sieve bed?)

One insight provided by the present disclosure is that the amount of oxygen to be produced and to be supplied to a patient 102 can be determined for controlling an amount of sieve beds in the adsorption units to switched on. Below is a table showing working conditions of two adsorption units in an integrated oxygen supply device in accordance with the present disclosure using formula 1: (need to define QO2, Qtotal, and FiO2 in the formula and the table below. Is FiO2 the desired FiO2?)

QO2=(FiO2−21%)/79%*Qtotal (Formula 1)

| Oxygen | | | | |
|---|---|---|---|---|
| Production Total | | QO2 | Adsorption Unit | Adsorption Unit |
| (LPM) | FiO2 | (LPM) | #1 On/Off | #2 On/Off |
| 10 | 25% | 0.51 | on | off |
| 10 | 30% | 1.14 | on | off |
| 10 | 40% | 2.41 | on | off |
| 10 | 50% | 3.67 | on | off |
| 20 | 45% | 6.08 | on | on |
| 30 | 35% | 5.32 | On | on |
| 30 | 40% | 7.22 | on | on |
| 40 | 30% | 4.56 | on | on |
| 40 | 35% | 7.09 | on | on |
| 50 | 25% | 2.53 | on | off |

In some implementation, the table shown above or similar tables (with more or less adsorption units) can be configured into the control unit 202 to control the working of oxygen concentration module 308. As can be seen, as oxygen total production goal is changed, for example—through an interface by an operator, the working state of the individual adsorption units can be controlled by the control unit 202 according to the table. For instance, when FiO2 is set to 25% for 10 LPM Oxygen production, only one adsorption unit is switched on by the control unit 202; and when FiO2 is set to 40% for 30 LPM Oxygen production, both adsorption units are switched on by the control unit 202.

Attention is now directed to the pressure valve 406. As shown in this example, in some implementation, the pressure valve 406 is used to push oxygen from a adsorption unit in the first state towards another adsorption unit in the second state for a given period of time to facilitate release of the N2 in the adsorption unit in the second state. For example, as illustration, thus not intended to be limiting, in the first period when the adsorption unit #1 404a produces the concentrated oxygen, the concentrated oxygen is allowed, by the pressure valve 406, to flow from the adsorption unit #1 404a to the adsorption unit #n 404n. This can help improve the release of the N2 in the adsorption unit #n 404n because the flown-in oxygen can expedite the release of the N2. Similarly, in the second period when the adsorption unit #n 404n is set to the first state to produce concentrated oxygen, the pressure valve 406, can be controlled by the control unit 202, to facilitate the oxygen to flow from the the adsorption unit #n 404n to the adsorption unit #1 404a to improve the N2 release therein.

It should be understood that the number of adsorption units that work in parallel and alternatively is a design choice and thus is not intended to be limited by the example shown in FIG. 4. For example, the adsorption unit #2 404b can be configured to work in parallel with the adsorption unit #1 404a or in parallel with adsorption unit #n 404n. This choice may be controlled dynamically by the control unit 202 according to a treatment plan, oxygen demand, entrainment factor and/or any other considerations.

In one implementation, the example 400 of the oxygen concentration module 308 comprises an oxygen detector 408 configured to detect a fraction of oxygen content in the concentrated oxygen produced. In this example, the oxygen detector 408 is arranged at an outlet path to the oxygen storage module 310 as shown. A concentration level of oxygen can be detected by the oxygen detector 408 such that when the concentration level of the oxygen in the starts to drop below a threshold, the control unit 202 can be triggered to improve the concentrated oxygen production-for example, by putting more adsorption units in parallel working states. The threshold level for the oxygen concentration level can be preset, for example, in the control unit 202, such that when the concentration level of the oxygen detected by the oxygen detector is determined to be below the preset oxygen concentration level, the control unit 202 is configured to generate a control signal to control the working states of the adsorption units in the example 400 of the oxygen concentration module 308.

In some embodiments, the oxygen concentration level may vary due to the decay of a material employed by a given adsorption unit in the integrated oxygen supply device. In those embodiments. the oxygen concentration is closed-loop controlled and monitored. In one embodiment, two oxygen concentration sensors #1 and #2 are installed in the example 400. The oxygen concentration sensor #11 is installed at the oxygen storage module 310 or its down stream before the blower. Oxygen concentration sensor #2 is installed at the down stream of the blending point. In that embodiment, based on sensor #1 feedback measurement, entrainment of air by the blower can be adjusted. When the generated oxygen concentration dropped, the flow control valve flow will be increased to maintain blended oxygen concentration. In that embodiment, oxygen concentration sensor #2 is used to monitor the blended oxygen concentration. This provides additional safety for blended oxygen concentration. If the oxygen sensor #1 fails or the oxygen flow sensor failed, it can be detected by the oxygen concentration sensor #2 in that case.

In some implementation, vacuum pressure swing adsorption (VPSA) oxygen generation is employed in the integrated oxygen supply device in accordance with disclosure. In those implementation, the VPSA oxygen generation involves vacuumed pressure swing adsorption. The nitrogen is released by negative pressure. In a VPSA system, the compressor 302 working pressure is lower than the PSA and less heat is generated.

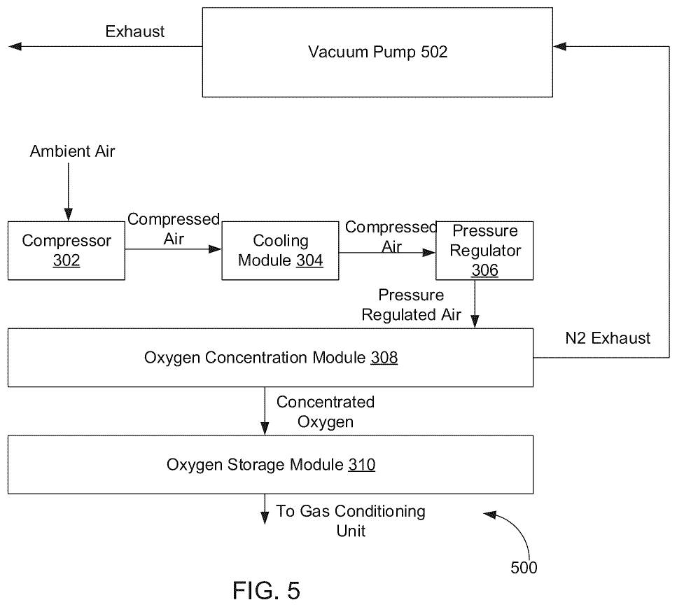

FIG. 5 illustrates another example 500 of an integrated oxygen supply device in accordance with the present disclosure. As can be seen, the example 500 of the integrated oxygen supply device includes a vacuum pump 502. As can be seen, the vacuum pump 502 is employed in this example to apply a vacuum to the oxygen concentration module 308 to facilitate the N2 exhaust in this example. In this way, together with the pressured gas provided to the oxygen concentration module 308, a vacuum pressure swing adsorption (VPSA) system is achieved. The example 500 provides a higher recovery of the adsorption units leading to a smaller compressor, blower, or other compressed gas or vacuum source and thus lower power consumption. Higher productivity of the example 500 also leads to smaller adsorption units. In one implementation, the working condition of the compressor 302 can be reduced to 25 PSIA and the working condition of the blower is set to 5 PSIA.

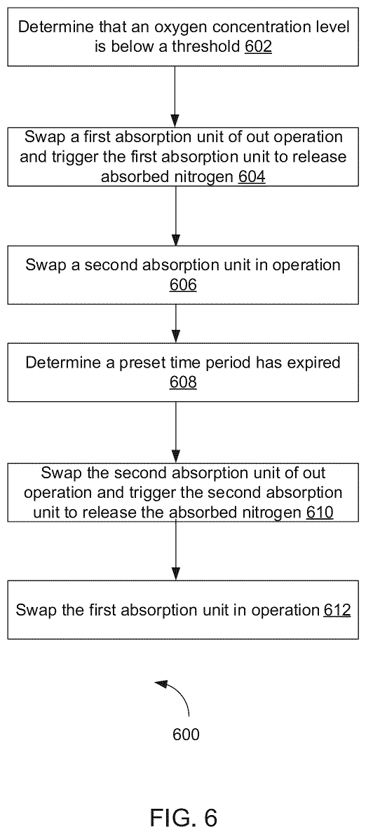

FIG. 6 illustrates an example method 600 for controlling the oxygen concentration module shown in FIG. 4. The operations of method 600 presented below are intended to be illustrative. In some embodiments, method 600 may be accomplished with one or more additional operations not described and/or without one or more of the operations discussed. Additionally, the order in which the operations of method 600 are illustrated in FIG. 6 and described below is not intended to be limiting.

In some embodiments, method 600 may be implemented by control unit 202 implemented by one or more of a processor, such as the ones shown in FIG. 2. The processor may include a digital processor, an analog processor, a digital circuit designed to process information, an analog circuit designed to process information, a state machine, and/or other mechanisms for electronically processing information. The control unit 202 may execute some or all of the operations of method 600 in response to instructions stored electronically on an electronic storage medium. The control unit 202 may include one or more components configured through hardware, firmware, and/or software to be designed for execution of one or more of the operations of method 600.

At 602, an oxygen concentration level produced by the oxygen concentration module is determined to below a threshold. In some embodiments, as mentioned, an oxygen detector can be arranged around an outlet of the oxygen concentration module to detect the oxygen concentration level produced by the oxygen concentration module. In implementations, readings of the oxygen concentration level detected by the oxygen detector can be stored in a storage. The readings can be obtained at 602 from time to time. The threshold may be preset or may be set dynamically by the control unit 202 according to a treatment plan, an oxygen demand by a patient, an entrainment goal, and/or any other consideration.

For example, a threshold of 90% can be set for the concentration level. However, it should be understood, the threshold used at 602 can be a design choice such that it can be set to control the oxygen concentration level. A higher concentration level would need a higher threshold and vice versa. The higher threshold would mean more adsorption units working in parallel in the oxygen concentration module at a given time period, which may increase the resource needed and thus cost for the oxygen production by the oxygen concentration module 308. However, on the other hand, higher oxygen concentration level would mean better oxygen enriched air supply to patient, which may be needed when the patient is in a worse-off condition demanding higher oxygen flow rate and concentration in the air supplied to the patient. In one implementation, the threshold used at 602 is adjustable by an operator. In that implementation an interface is provided to facilitate the operator to adjust the threshold to control the oxygen concentration level of the oxygen enriched air released by the air separation unit.

In some embodiments, the threshold level at 602 can be dynamically adjusted through an interface provided by the integrated oxygen supply device. For example, the interface may be configured to enable an operator of the integrated oxygen supply device to select a preset oxygen supply mode, which may correspond to a range of oxygen concentration level in the enriched oxygen air. For example, an oxygen supply mode corresponding to an oxygen concentration level between 26%-30% may be provided to assist the patient breathe. As will be described, this can help improve an efficiency and power consumption of the integrated oxygen supply device.

At 604, a first adsorption unit is swapped out of operation. For example, this can be achieved by sweeping a switch valve connected to the first adsorption unit to the second state as mentioned and illustrated herein. In the second state, the first adsorption unit is set to release absorbed N2 to the environment, which can improve oxygen production efficacy of the first adsorption unit.

At 606, a second adsorption unit is swapped into operation. Although, in this example, 604 and 606 are illustrated in sequence, in implementation, these two operations may take place simultaneously or nearly simultaneously. For example, the control unit 202 can sweep a switch valve the second adsorption unit to the first state mentioned and illustrated herein. In the first state, the switch valve facilitates the second adsorption unit to receive pressure regulated air to produce concentrated oxygen. In implementations, multiple adsorption units can be set to work in parallel with the second adsorption unit to increase the oxygen concentration level.

At 608, a preset time period is determined to have expired. For instance, the preset time period may be 6 seconds. At 610, the second adsorption unit is swapped out of the operation because the preset time period has expired. As mentioned, the preset time period may be used to control an operation duration of the second adsorption unit after it is swapped into operation. For example, the control unit 202 can be configured to sweep the switch valve connected to the second adsorption unit to the second state to trigger the second adsorption unit to release absorbed nitrogen.

At 612, the first adsorption unit is swapped into operation. Although, in this example, 610 and 612 are illustrated in sequence, in implementation, these two operations may take place simultaneously or nearly simultaneously. For example, the control unit 202 can be configured to sweep the switch valve connected to the first adsorption unit to the first state to trigger the first adsorption unit to absorb nitrogen from the received air and thus produce concentrated oxygen.

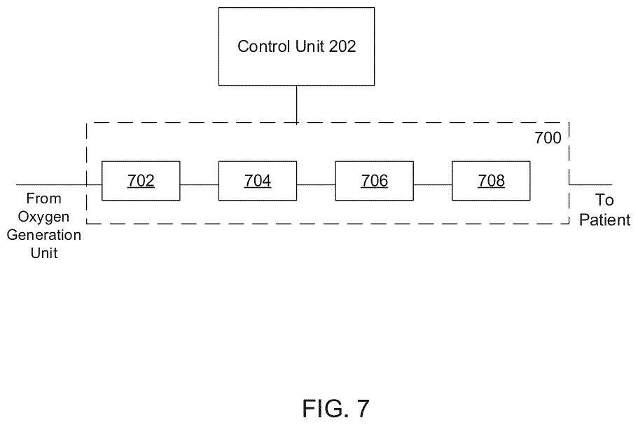

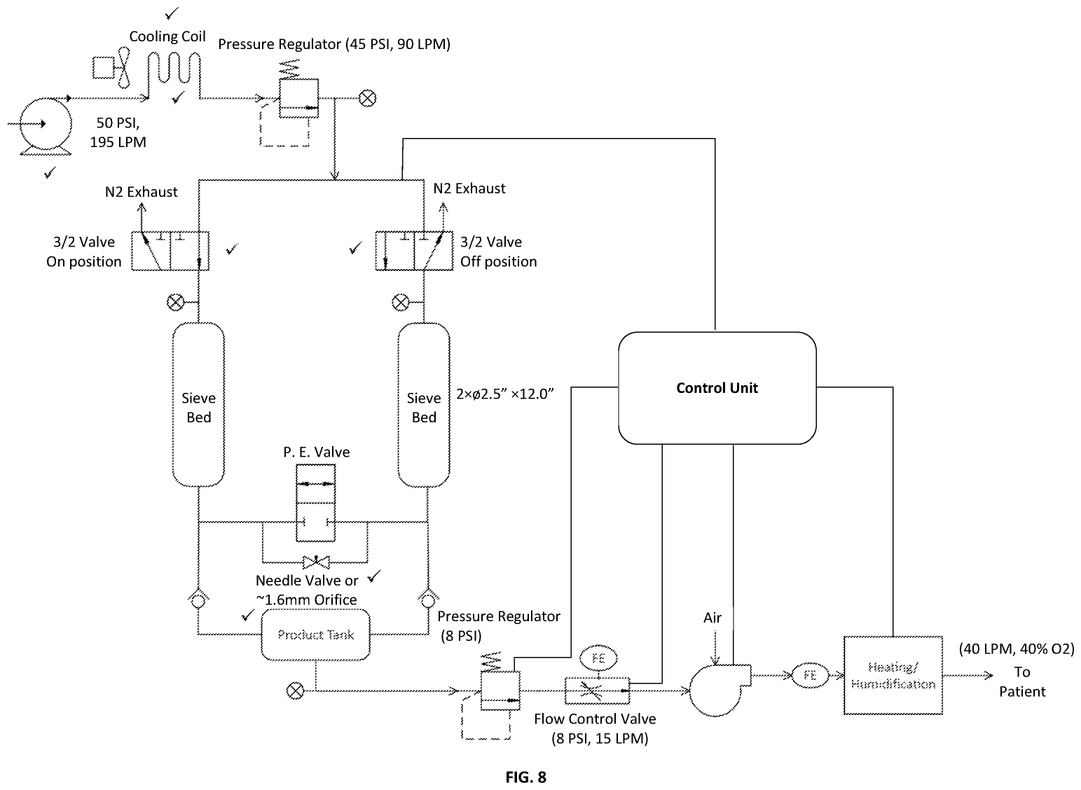

FIG. 7 illustrates one example 700 of a gas conditioning unit 206 shown in FIG. 2. In this example, the example 700 of the gas conditioning unit 206 comprises a pressure regulator 702, a flow control valve 704, an air blower 707, a heating/humidification unit 708, and/or any other components. The pressure regulator 702 is configured to adjust the concentrated oxygen from the oxygen generation unit 204 to a desired pressure level. In one embodiment, the desired pressure level is 8 PSI and is controlled by the control unit 202. In implementation, this adjustment can be controlled by the control unit 202 dynamically according to the pressure level of the concentrated oxygen produced by the oxygen generation unit 204. For instance, a sensor can be arranged to detect the pressure level of the concentrated oxygen level.

The flow control valve 704 is controlled by the control unit 202 to adjust the flow rate of the concentrated oxygen produced by the oxygen generation unit 204 to a desired flow rate. In one embodiment, the desired pressure level is 15 LPM and is controlled by the control unit 202. In implementation, this adjustment can be controlled by the control unit 202 dynamically according to the flow rate of the concentrated oxygen produced by the oxygen generation unit 204. For instance, a sensor can be arranged to detect the flow rate level of the concentrated oxygen level. In some embodiments, the flow control valve 704 operates in two modes. One is continuous flow mode, where the flow is adjusted according to patient demand flow. The other one is pulsation mode, where the pulsation of oxygen is released to the air blower 706. In implementation, the pulsation amplitude can be controlled by the control unit 202 by measuring oxygen flow for each pulse and adjust the electrical power to the valve.

The air blower 706 is configured to receive ambient air and the pressure and flow rate regulated oxygen, and to output blended air at a desired FiO2 level, for example 40/40. The air blower 706 is configured to combine ambient air with concentrated oxygen produced by the oxygen generation unit 204 at a desired FiO2 ratio. The desired FiO2 ratio may be determined to achieve a final oxygen fraction delivered to the patient. In one embodiment, the desired ration is determined by the patient physiological profile and treatment requirement.

In some embodiments, the flow rate of the ambient air and the flow rate of the oxygen injected into the air blower 706 are independently controlled by the control unit 202 before they are blended in the air blower 706. For example, the control unit 202, in one implementation, is configured to adjust an oxygen concentration level of the blended air by adjusting the flow rate of the oxygen or the flow rate of the ambient art. In some implementations, a sensor is arranged at an outlet of the air blower 706 to monitor the oxygen concentration level from the air blower 706 and the monitored concentration level is reported to the control unit 202 from time to time to facilitate the control unit 202 to adjust the oxygen concentration level in the blended air. For example, the control unit 202 is configured to operate the oxygen generation unit 204 according to a desired FiO2 level for the air blended at the air blower 706. For instance, the control unit 202 can control a flow rate of the oxygen from the oxygen generation unit for achieving the desired FiO2. As mentioned, the control unit 202 is configured to achieve the desire FiO2 for the blended air by independently controlling the ambient air flow rate into the air blower 706.

In some embodiments, the control unit 202 is configured to control a flow rate of the ambient air and a flow rate of oxygen to achieve a blended ratio of for a blended air without using the air blower 706. In those embodiments, a valve may be employed to combine the ambient air and the oxygen. In some implementations, the ambient air may be flown in one passage way and the oxygen may be flown in another passage way, such as two different tubes. In those embodiments, the ambient air and oxygen are combined to achieve a blended air. In those embodiments, the control unit 202 is configured to achieve a desired FiO2 for the blended air. For example, the control unit 202 is configured control a flow rate of the ambient air and a flow rate of the oxygen.

The heating/humidification unit 708 is configured to heat the gas to a desired temperature point. The temperature of the inhaled gas by a patient impacts the comfort level of the patient 102 and subsequently the compliance of the treatment. The inhaled gas temperature also impacts the physiological response of the patient's respiratory system. The gas temperature impacts an achievable humidification level of the inhaled gas, which subsequently impacts the treatment effectiveness and potential side effect of the treatment. Thus, accurate temperature control is important to ensure that the temperature of the inhaled gas at the nasal cannula. Typically, the temperature of the inhaled gas to a patient is set at 37 degrees or slightly lower, supported by prior clinical studies. An advantage of the heating/humidification unit 708 is the air conditioning is achieved through the control unit 202 adjusting the heating/humidification unit 708 rather than through some conventional methods, such as through a membrane.

The heating/humidification unit 708 is configured to humidify the heated gas. Normally, the upper respiratory tract of a healthy person humidifies the inhaled air to saturation level at body temperature, which is close to 37 degrees Celsius (37° C.). If natural respiratory humidification fails, pulmonic infections and damage to lung tissue may be the consequence. The appropriate humidification level is essential for the healthy functioning of the human respiratory system and effective treatment of the breathing machine. Since the flow rate of the invented breathing device is significantly larger than the normal inspiration flow rate accustomed to the human respiratory system, the natural respiratory system may not be able to provide sufficient humidification needed. Thus, it is essential to elevate the humidity level of the heated gas to alleviate the burden to the human respiratory system when using the breathing machine.

In various embodiments, the added humidity and heat to the blended gas by the gas conditioning unit 206 makes inhaled gas delivered to the patient more palatable, especially at a higher flow rate. The most effective humidification is the pass-through type humidification, in which the gas pass on top heated water, and additional water vapor is added and the gas is also heated by thermo energy transferred from the warm water. An electrical heating electrical heating element is used to heat up water and a closed-loop temperature/humidity control is employed. The process is energy hungry, due to the thermo energy dissipating and loss in the head conductivity. In one embodiment, a heated water reservoir is applied as the humidification subunit 2078 and the heating subunit 2076 in the integrated oxygen supply device 100. In that embodiment, the temperature of the reservoir is set at a pre-determined value, based on the temperature humidity lookup table.

In another embodiment, temperature sensors and humidity sensors are arranged detect the temperature and humidity of the inhaled gas at or near a nasal cannula to the patient. In that embodiment, feedback control mechanism is applied in the control unit 202 to control heating/humidification unit 708 to ensure achievement of the desired temperature and humidity levels of the inhaled gas at the nasal cannula.

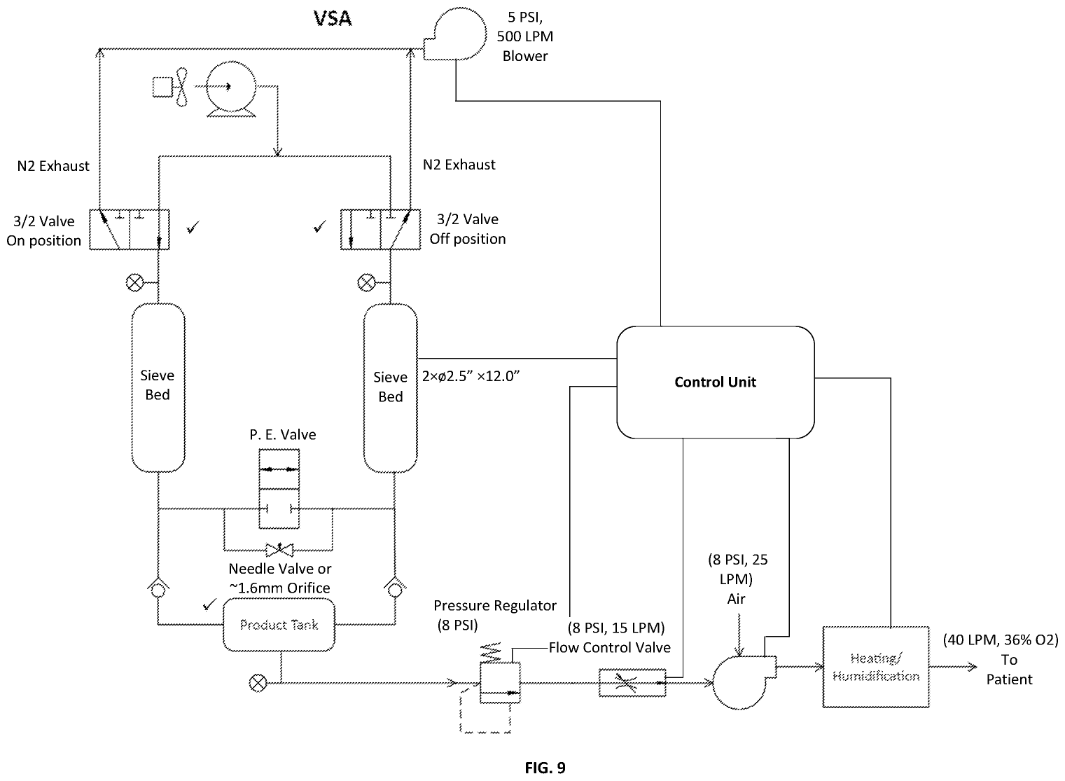

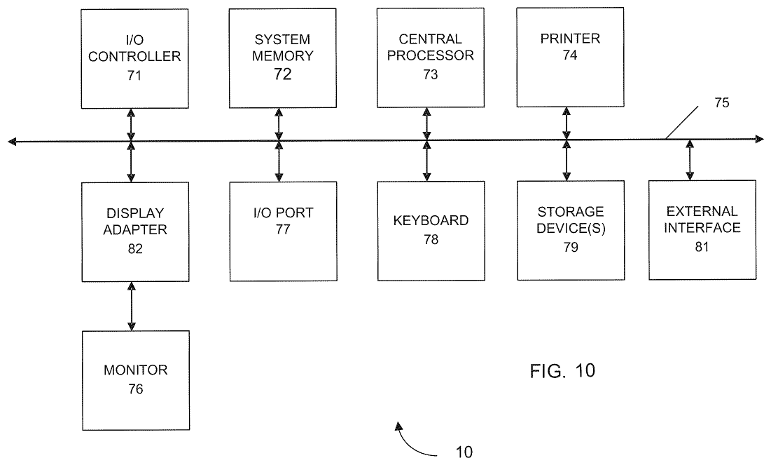

FIG. 8 illustrates another example implementation of an integrated oxygen supply device 800 in accordance with the present disclosure. As can be seen, the integrated oxygen supply device 800 comprises two sieve beds working alternatively controlled by the control unit. In this example, the switch valves are controlled by the control unit. The switching valves are controlled to sweep at 5 seconds interval. At a given time, one sieve bed is working at the oxygen generation state. In this example, at 50 PSI, The N2 is absorbed by a working sieve bed, and the concentrated oxygen is produced and stored in product tank. The non-working sieve bed is in a recycle state—where, the switch valve is open to the ambient, and the nitrogen is released from non-working sieve bed.

In some embodiments, the integrated oxygen supply device 800 has a reduced dimension. In one embodiment, the integrated oxygen supply device has a dimension of 15(L)*8 (H)*19 (W) inches, capable of producing gas at 40/40 FiO2 and weigh less than 30 pound.

FIG. 9 illustrates another example of an integrated oxygen supply device in accordance with the present disclosure. In this example 900, as can be seen, a vacuum pump is added in the circuits to apply vacuum to the exhausted N2 from the sieve beds. In this way, a VPSA is implemented in the example 900.

### Example Computer System

Any of the computer systems mentioned herein may utilize any suitable number of subsystems. Examples of such subsystems are shown in FIG. 8 in computer system 10, which can be configured to implement various features and/or functions described herein. In some embodiments, a computer system includes a single computer apparatus, where the subsystems can be the components of the computer apparatus. In other embodiments, a computer system can include multiple computer apparatuses, each being a subsystem, with internal components.

The subsystems shown in FIG. 8 are interconnected via a system bus 75. Additional subsystems such as a printer 74, keyboard 78, storage device(s) 79, monitor 76, which is coupled to display adapter 82, and others are shown. Peripherals and input/output (I/O) devices, which couple to I/O controller 71, can be connected to the computer system by any number of means known in the art such as input/output (I/O) port 77 (e.g., USB, FireWire). For example, I/O port 77 or external interface 81 (e.g. Ethernet, Wi-Fi, etc.) can be used to connect computer system 10 to a wide area network such as the Internet, a mouse input device, or a scanner. The interconnection via system bus 75 allows the central processor 73 to communicate with each subsystem and to control the execution of instructions from system memory 72 or the storage device(s) 79 (e.g., a fixed disk, such as a hard drive or optical disk), as well as the exchange of information between subsystems. The system memory 72 and/or the storage device(s) 79 may embody a computer readable medium. Any of the data mentioned herein can be output from one component to another component and can be output to the user.

A computer system can include a plurality of the same components or subsystems, e.g., connected together by external interface 81 or by an internal interface. In some embodiments, computer systems, subsystem, or apparatuses can communicate over a network. In such instances, one computer can be considered a client and another computer a server, where each can be part of a same computer system. A client and a server can each include multiple systems, subsystems, or components.

It should be understood that any of the embodiments of the present invention can be implemented in the form of control logic using hardware (e.g. an application specific integrated circuit or field programmable gate array) and/or using computer software with a generally programmable processor in a modular or integrated manner. As used herein, a processor includes a single-core processor, multi-core processor on a same integrated chip, or multiple processing units on a single circuit board or networked. Based on the disclosure and teachings provided herein, a person of ordinary skill in the art will know and appreciate other ways and/or methods to implement embodiments of the present invention using hardware and a combination of hardware and software.

Any of the software components or functions described in this application may be implemented as software code to be executed by a processor using any suitable computer language such as, for example, Java, C, C++, C #, Objective-C, Swift, or scripting language such as Perl or Python using, for example, conventional or object-oriented techniques. The software code may be stored as a series of instructions or commands on a computer readable medium for storage and/or transmission, suitable media include random access memory (RAM), a read only memory (ROM), a magnetic medium such as a hard-drive or a floppy disk, or an optical medium such as a compact disk (CD) or DVD (digital versatile disk), flash memory, and the like. The computer readable medium may be any combination of such storage or transmission devices.

Such programs may also be encoded and transmitted using carrier signals adapted for transmission via wired, optical, and/or wireless networks conforming to a variety of protocols, including the Internet. As such, a computer readable medium according to an embodiment of the present invention may be created using a data signal encoded with such programs. Computer readable media encoded with the program code may be packaged with a compatible device or provided separately from other devices (e.g., via Internet download). Any such computer readable medium may reside on or within a single computer product (e.g. a hard drive, a CD, or an entire computer system), and may be present on or within different computer products within a system or network. A computer system may include a monitor, printer, or other suitable display for providing any of the results mentioned herein to a user.

Any of the methods described herein may be totally or partially performed with a computer system including one or more processors, which can be configured to perform the steps. Thus, embodiments can be directed to computer systems configured to perform the steps of any of the methods described herein, potentially with different components performing respective steps or a respective group of steps. Although presented as numbered steps, steps of methods herein can be performed at a same time or in a different order. Additionally, portions of these steps may be used with portions of other steps from other methods. Also, all or portions of a step may be optional. Additionally, any of the steps of any of the methods can be performed with modules, circuits, or other means for performing these steps.

The specific details of particular embodiments may be combined in any suitable manner without departing from the spirit and scope of embodiments of the invention. However, other embodiments of the invention may be directed to specific embodiments relating to each individual aspect, or specific combinations of these individual aspects.

The above description of exemplary embodiments of the invention has been presented for the purposes of illustration and description. It is not intended to be exhaustive or to limit the invention to the precise form described, and many modifications and variations are possible in light of the teaching above. The embodiments were chosen and described in order to best explain the principles of the invention and its practical applications to thereby enable others skilled in the art to best utilize the invention in various embodiments and with various modifications as are suited to the particular use contemplated.

A recitation of “a”, “an” or “the” is intended to mean “one or more” unless specifically indicated to the contrary. The use of “or” is intended to mean an “inclusive or,” and not an “exclusive or” unless specifically indicated to the contrary.

Two states are also for illustration purpose. It is possible for an embodiment where remote command and local command for changing oxygen device settings be accepted without state switches.

All patents, patent applications, publications, and descriptions mentioned herein are incorporated by reference in their entirety for all purposes. None is admitted to be prior art.

Having described several example configurations, various modifications, alternative constructions, and equivalents may be used without departing from the spirit of the disclosure. For example, the above elements may be components of a larger system, wherein other rules may take precedence over or otherwise modify the application of the invention. Also, a number of steps may be undertaken before, during, or after the above elements are considered.

What is claimed is:

1. An oxygen supply device comprising:
an oxygen production unit, a control unit, an air blower and a gas conditioning unit, wherein
the oxygen production unit comprises multiple adsorption units including a first adsorption unit and a second adsorption unit;
the air blower is configured to receive ambient air and oxygen generated by the oxygen production unit and output blended air at a desired FiO2 ratio;
the gas conditioning unit is connected to the air blower and is configured to generate conditioned air using the blended air from the air blower; and, wherein the control unit is configured to:
control the first and second adsorption unit to operate independently from each other such that first and second adsorption units operate alternatively; and
independently control a flow rate of the oxygen generated by the oxygen generation unit and a flow rate of ambient air into the air blower.

2. The oxygen supply device of claim 1, wherein the first adsorption unit is connected to a first valve; and wherein controlling the first adsorption unit to operate includes controlling the first valve to sweep to a first position, at which a circuit between a pressure regulator and the first adsorption unit is connected.

3. The oxygen supply device of claim 1, wherein the first adsorption unit is connected to a first valve; and wherein controlling the first adsorption unit to operate includes controlling the first valve to sweep to a second position, at which a circuit between a vacuum pump and the first adsorption unit is connected.

4. The oxygen supply device of claim 2, wherein the control unit is further configured to:
determine that a concentration level of the oxygen produced by the oxygen production unit is below a threshold; and
control the first valve to swipe to the second position in response to the determination that the concentration level of the oxygen produced by the oxygen production unit is below the threshold, wherein when the first valve is in the second position, the adsorption unit is facilitated to separate oxygen and nitrogen.

5. The oxygen supply device of claim 1, wherein the control unit is configured to control the first and second adsorption units to operate in parallel.

6. The oxygen supply device of claim 1, wherein the oxygen production unit further comprises a pressure valve connected to the adsorption unit; and, wherein the control unit is configured to control the pressure valve to facilitate concentrated oxygen produced by the adsorption unit to be released to at least one another adsorption unit in the oxygen supply device.

7. The oxygen supply device of claim 1, wherein a gas produced by the oxygen supply device has an oxygen concertation level at least higher than 21% and/or a flow rate higher 10 liter per minute.

8. An oxygen production unit comprising:
an oxygen generation module, an air blower and a control unit, wherein
the oxygen generation module comprises multiple adsorption units including a first adsorption unit and a second adsorption unit;
the air blower is configured to receive ambient air and oxygen generated by the oxygen production unit and output blended air at a desired FiO2 ratio; and
the control unit is configured to:
control the first and second adsorption unit to operate independently from each other such that first and second adsorption units operate alternatively; and
independently control a flow rate of the oxygen generated by the oxygen generation module and a flow rate of ambient air into the air blower.

9. The oxygen production unit of claim 8, wherein the first adsorption unit is connected to a first valve; and wherein controlling the first adsorption unit to operate includes controlling the first valve to sweep to a first position, at which a circuit between a pressure regulator and the first adsorption unit is connected.

10. The oxygen production unit of claim 8, wherein the first adsorption unit is connected to a first valve; and wherein controlling the first adsorption unit to operate includes controlling the first valve to sweep to a second position, at which a circuit between a vacuum pump and the first adsorption unit is connected.

11. The oxygen production unit of claim 9, wherein the control unit is further configured to:
determine that a concentration level of the oxygen produced by the oxygen production unit is below a threshold; and
control the first valve to swipe to the second position in response to the determination that the concentration level of the oxygen produced by the oxygen production unit is below the threshold, wherein when the first valve is in the second position, the adsorption unit is facilitated to separate oxygen and nitrogen.

12. The oxygen production unit of claim 8, wherein the control unit is configured to control the first and second adsorption units to operate in parallel.

13. The oxygen production unit of claim 8, wherein the oxygen production unit further comprises a pressure valve connected to the adsorption unit; and, wherein the control unit is configured to control the pressure valve to facilitate concentrated oxygen produced by the adsorption unit to be released to at least one another adsorption unit in the oxygen production unit.

14. The oxygen production unit of claim 8, wherein a gas produced by the oxygen production unit has an oxygen concertation level at least higher than 21% and/or a flow rate higher 10 liter per minute.

15. An oxygen supply device comprising:
a control unit, an air blower and a gas conditioning unit, wherein
the gas conditioning unit comprises a pressure regulation unit connected to the oxygen supply device; and, wherein the control unit is configured to:
control the pressure regulation unit to adjust a pressure of oxygen produced by the oxygen supply device; and
independently control a flow rate of the oxygen and a flow rate of ambient air into the air blower.

16. The oxygen supply device of claim 15, further comprises oxygen concentration module comprising multiple adsorption units including a first and second adsorption units; and, wherein the control unit is further configured to control the first and second adsorption units to operate alternatively

17. The oxygen supply device of claim 16, wherein the first adsorption unit is connected to a first valve; and wherein controlling the first adsorption unit to operate includes controlling the first valve to sweep to a second position, at which a circuit between a vacuum pump and the first adsorption unit is connected.

18. The oxygen supply device of claim 16, wherein the control unit is configured to control the first and second adsorption units to operate in parallel.

19. The oxygen supply device of claim 1, wherein the oxygen production unit further comprises a pressure valve connected to the adsorption unit; and, wherein the control unit is configured to control the pressure valve to facilitate concentrated oxygen produced by the adsorption unit to be released to at least one another adsorption unit in the oxygen supply device.
